(19) United States
(12) Patent Application Publication
(10) Pub. No.: US 2024/0090806 A1
(43) Pub. Date: Mar. 21, 2024

(54) Arrangement of Wrist-Side Continuous Electrodermal Activity Electrodes on a Wearable Device for Detecting Stress Events

(71) Applicant: Fitbit, Inc., San Francisco, CA (US)

(72) Inventors: Lindsey Sunden, San Francisco, CA (US); Daniel Steven Howe, San Diego, CA (US); Conrad Guanchung Wang, Tustin, CA (US); Ryotaro Miyagawa, Burlingame, CA (US); Seamus David Thomson, Mountain View, CA (US); David Duncanson Gutschick, Santa Clara, CA (US)

(21) Appl. No.: 18/013,704

(22) PCT Filed: Aug. 23, 2021
(86) PCT No.: PCT/US2021/047119

§ 371 Date: Dec. 29, 2022

(51) Int. Cl. A61B 5/16; A61B 5/00; A61B 5/0531

(52) U.S. Cl. CPC A61B 5/165; A61B 5/0531; A61B 5/681; A61B 5/7282; A61B 5/742; A61B 2562/046

(57) ABSTRACT

A wearable computing device includes a housing having a wrist-side face configured to sit against a wrist of a user of the wearable computing device when being worn by the user, an electronic display arranged within the housing, a plurality of biometric sensor electrodes positioned on the wrist-side face so as to maintain skin contact with the user when being worn on the wrist by the user, and at least one driver communicatively coupled to the plurality of biometric sensor electrodes. Each of the plurality of biometric sensor electrodes continuously measures, at least, one or more parameters indicative of electrical impedance of the user at a location of the skin contact. Further, the wearing computing device includes at least one controller(s) communicatively coupled to the plurality of biometric sensor electrodes and the driver and is configured to determine skin conductance, changes to the skin conductance, a skin conductance level, SCL, and/or skin conductance responses, SCRs, of the user over a certain time period using the electrical impedance of the user.

### FIELD

The present disclosure relates generally to wearable computing devices, and more particularly, to an arrangement of wrist-side continuous electrodermal activity sensor electrodes on a wearable device, thereby providing increased contact area and improved signal quality of the sensor signals.

### BACKGROUND

Recent consumer interest in personal health has led to a variety of personal health monitoring devices being offered on the market. Recent advances in sensor, electronics, and power source miniaturization have allowed the size of personal health monitoring devices, also referred to herein as “biometric tracking” or “biometric monitoring” devices, to be offered in extremely small sizes that were previously impractical.

These biometric monitoring devices may collect, derive, and/or provide one or more of the following types of information: heart rate, calorie burn, floors climbed and/or descended, location and/or heading, elevation, ambulatory speed and/or distance traveled, etc. Recent advances in technology, including those available through consumer devices, have provided for corresponding advances in health detection and monitoring. For example, devices such as fitness trackers and smartwatches are able to determine information relating to the pulse or motion of a person wearing the device. Due to capabilities of conventional devices, however, the amount and types of health information able to be determined using such devices has been limited.

However, recent advances in sensor, electronics, and power source miniaturization have allowed the size of personal health monitoring devices to be offered in extremely small sizes that were previously impractical, thereby allowing for additional parameters to be monitored. As one example, certain biometric monitoring devices measure electrodermal activity, EDA, responses, which are tiny electrical changes on the user's skin, using an electrical sensor to detect EDA responses through the palm of the user's hand.

In particular, for EDA responses, electrical impedance is measured through the palm and skin conductance is calculated based on the measured electrical impedance. Skin conductance responses, SCRs, are then determined from the calculated skin conductance, which are the spikes in the calculated skin conductance data. More specifically, to identify SCR spikes, the skin conductance is compared to a baseline value or point of reference. In general, SCRs are more accurately identified using data collected from certain regions of the human body that are known to have a high sweat gland density, such as a user's palm.

However, there are two primary electrodermal activity features that can be evaluated from skin conductance: 1) SCRs (as previously discussed), and 2) skin conductance level. SCL. SCL, rather than SCRs alone, can be beneficial in determining a user's continuous electrodermal activity, cEDA, as cEDA can be used as a precursor for certain biological events, such as the body's response to acute stress events. However, cEDA can be difficult to detect using electrodes that are mounted on a top face of a biometric monitoring device (i.e., on a non-body contacting surface) as cEDA needs continuous skin contact to provide accurate readings. Further, in some instances, FDA devices may require active interaction from the user.

In some instances, devices may include ventral wrist-side FDA measurements, however, there are drawbacks to such devices, including but not limited to, the electrical connections having to go from the device body through the wristband to the electrodes, the electrodes having to protrude from the wristband to maintain constant contact, and the muscles and tendons being used for a tight grip pass under the electrodes, which can lead to erroneous changes in the baseline measurements.

Accordingly, the present disclosure is directed to a wearable biometric monitoring device having a dorsal wrist-side arrangement of cEDA electrodes. In particular, the present disclosure is directed to the layout, sizing, spacing, and composition of the dorsal wrist-side cEDA electrodes on a wearable biometric monitoring device for detecting acute stress events. The present disclosure also addresses the challenges associated with obtaining sufficient signal quality when electrodes are placed on the wrist-side of the wearable biometric monitoring device.

### SUMMARY

Aspects and advantages of embodiments of the present disclosure will be set forth in part in the following description, or can be learned from the description, or can be learned through practice of the embodiments.

One example aspect of the present disclosure is directed to a wearable computing device. The wearable computing device includes a housing having a dorsal wrist-side face configured to sit against a dorsal wrist of a user of the wearable computing device when being worn by the user, an electronic display arranged within the housing, a plurality of biometric sensor electrodes positioned on the wrist-side face so as to maintain skin contact with the user when being worn on the wrist by the user, and at least one driver communicatively coupled to the plurality of biometric sensor electrodes. Each of the plurality of biometric sensor electrodes measures, at least, one or more parameters indicative of electrical impedance of the user at a location of the skin contact. Further, the driver is communicatively coupled to at least one controller(s). Moreover, the controller(s) is configured to determine a skin conductance level, SCL, of the user over a certain time period based on the electrical impedance of the user and calculate a stress state of the user based, at least in part, on the SCL.

Another example aspect of the present disclosure is directed to a computer-implemented method of monitoring a stress state of a user using a wearable computing device. The wearable computing device includes a plurality of biometric sensor electrodes on a dorsal wrist-side face of a housing of the wearable computing device. The computer-implemented method includes placing one or more of the plurality of biometric sensor electrodes adjacent to a dorsal wrist of the user. Further, the method includes continuously measuring, via the one or more of the plurality of biometric sensor electrodes of the wearable computing device, at least, one or more parameters indicative of electrical impedance of the user at the wrist over a certain time period. Moreover, the method includes determining, via at least one controller of the wearable computing device, a skin conductance level, SCL, of the user over the certain time period based on the electrical impedance of the user. In addition, the method includes calculating, via the controller(s), the stress state of the user based, at least in part, on the SCL. Thus, the method further includes displaying, via a display of the wearable computing device, the stress state to the user.

Other aspects of the present disclosure are directed to various systems, apparatuses, non-transitory computer-readable media, user interfaces, and electronic devices.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

Detailed discussion of embodiments directed to one of ordinary skill in the art is set forth in the specification, which makes reference to the appended figures, in which:

FIG. 1 provides a graphical representation of electrodermal activity, FDA, amplitude (y-axis) versus time (x-axis) according to one embodiment of the present disclosure;

FIG. 2 provides a perspective view of a wearable computing device on a dorsal wrist of a user according to one embodiment of the present disclosure;

FIG. 3 provides a front perspective view of a wearable computing device according to one embodiment of the present disclosure;

FIG. 4 provides a rear perspective view of the wearable computing device of FIG. 3;

FIG. 5 provides an exploded view of the display of the wearable computing device of FIG. 3;

FIG. 6 illustrates various controller components of an example system that can be utilized according to one embodiment of the present disclosure;

FIG. 7 provides a schematic diagram of an example set of devices that are able to communicate according to one embodiment of the present disclosure;

FIGS. 8A-8D provides various embodiments of a layout of the plurality of biometric sensor electrodes on a wrist-side of the wearable computing device according to the present disclosure;

FIGS. 9A-9L provides still further embodiments of a layout of the plurality of biometric sensor electrodes on a wrist-side of the wearable computing device according to the present disclosure;

FIG. 10 illustrates a wrist-side view of the wearable computing device according to the present disclosure, particularly illustrating an arrangement of the plurality of biometric sensor electrodes with respect to an elbow and a wrist of a user;

FIG. 11 illustrates a graphical representation of EDA amplitude (i.e. admittance magnitude calculated as the square root of the sum of the conductance squared and the susceptance, squared) (y-axis) versus time (x-axis) according to one embodiment of the present disclosure, particularly illustrating the impact of certain movements on the EDA measurements over time;

FIGS. 12A-12B illustrates graphical representations of admittance, ambient humidity, and temperature (e.g., skin or ambient temperature according to one embodiment of the present disclosure, particularly illustrating the impact of humidity and temperature on the conductivity as measured by a wearable computing device worn on a wrist of a user; and

FIG. 13 illustrates a flow diagram of one embodiment of a method of monitoring a stress state of a user using a wearable computing device according to the present disclosure.

### DETAILED DESCRIPTION

Reference now will be made in detail to embodiments of the invention, one or more examples of which are illustrated in the drawings, Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

### Overview

Recent advances in technology, including those available through consumer devices, have provided for corresponding advances in health detection and monitoring. For example, devices such as fitness trackers and smartwatches are able to determine information relating to the pulse or motion of a person wearing the device. Due to capabilities of conventional devices, however, the amount and types of health information able to be determined using such devices has been limited.

However, recent advances in sensor, electronics, and power source miniaturization have allowed the size of personal health monitoring devices to be offered in extremely small sizes that were previously impractical. For example, certain biometric monitoring devices include a wristband having a housing that is about 4 centimeters (cm) wide by 4 cm long by 1.3 cm thick. Such biometric monitoring devices generally include a display, battery, sensors, electronics package, wireless communications capability, power source, and an interface button packaged within this small volume. Moreover, certain biometric monitoring devices include a variety of sensors for measuring multiple biological parameters that can be beneficial to a user of the device, such as a heart rate sensor, multi-purpose electrical sensors compatible with electrocardiogram. ECG, and EDA applications, red and infrared sensors, a gyroscopes, an altimeter, an accelerometer, a temperature sensor, an ambient light sensor, Wi-Fi, GPS, a vibration or haptic feedback sensor, a speaker, and a microphone, among others. As one example, certain biometric monitoring devices measure EDA responses, which are changes in the conductance and susceptance between the electrodes on the user's skin, typically using a single-path electrical sensor to detect EDA responses through the palm of the user's hand.

For example, for EDA responses, electrical impedance is measured through the palm or the ventral side of the user's fingers, and skin conductance is calculated based on the measured electrical impedance. Skin conductance responses, SCRs, are then determined from the calculated skin conductance, which are the spikes in the calculated skin conductance data. More specifically, to identify SCR spikes, the skin conductance is compared to a baseline value or point of reference. In general, SCRs are more accurately determined from data collected from a user's palm or the ventral side of the user's fingers.

However, there are two primary electrodermal activity features that can be evaluated from skin conductance: 1) SCRs (as previously discussed), and 2) SCL. SCL, rather than SCRs alone, can be beneficial in determining a user's continuous electrodermal activity, cEDA, as cEDA can be used as an indicator of certain biological events, such as the body's response to acute stress events. However, cEDA can be difficult to detect using electrodes that are mounted on a top face of a biometric monitoring device on a non-body contacting surface) as cEDA needs continuous and stable skin contact to provide accurate readings.

More particularly, in terms of timing, the difference between the two is that SCRs occur on the scale of seconds, whereas SCL is evaluated across seconds, minutes, and/or hours. As an example, FIG. 1 illustrates a graphical representation 10 of EDA amplitude versus tune in milliseconds (ms). As shown, the graphical representation 10 provides a comparison of phasic skin conductance response, SCR, 12, the tonic skin conductance level, SCL, 14, and the EDA peaks 16 to depict differences between SCRs and SCL. Thus, as shown via the graphical representation 10 of FIG. 1, for changes in SCI, to be accurately detected, skin conductance needs to be continuously measured (over minutes/hours/days). However, with palm or finger measurements used to measure EDA, an accurate SCL is difficult to determine since a user would be required to continuously hold his or her palm or hand against a wearable device.

Accordingly, the present disclosure is directed to a wearable biometric monitoring device having a dorsal wrist-side arrangement of cEDA electrodes. In particular, the present disclosure is directed to the layout, sizing, spacing, and composition of the dorsal wrist-side cEDA electrodes on a wearable biometric monitoring device for detecting acute stress events. The present disclosure also addresses the challenges associated with obtaining sufficient signal quality when electrodes are placed on the wrist-side of the wearable biometric monitoring device.

In accordance with embodiments described herein, a configuration is proposed according to which increased contact area, an improved manner of maintaining skin contact, and correspondingly improved signal quality of the sensor signals can be ensured.

With reference now to the Figures, example embodiments of the present disclosure will be discussed in further detail.

### Example Devices and Systems

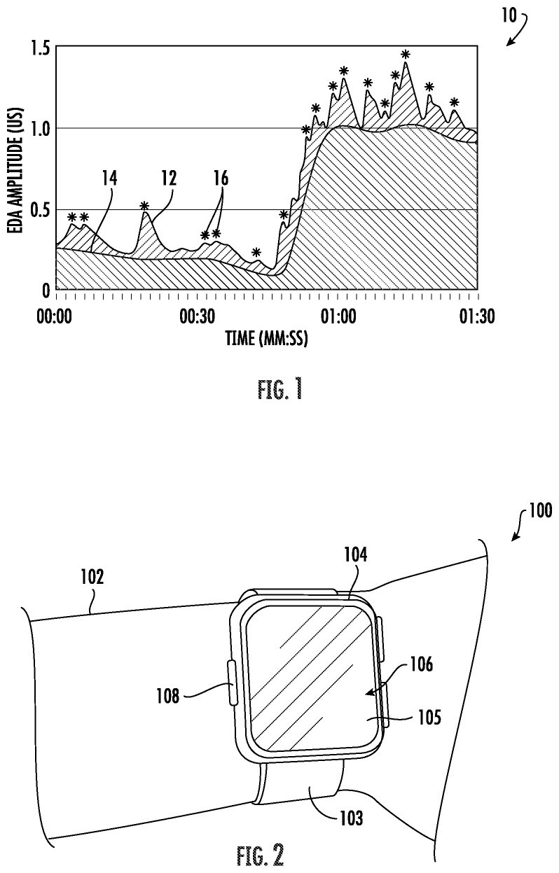

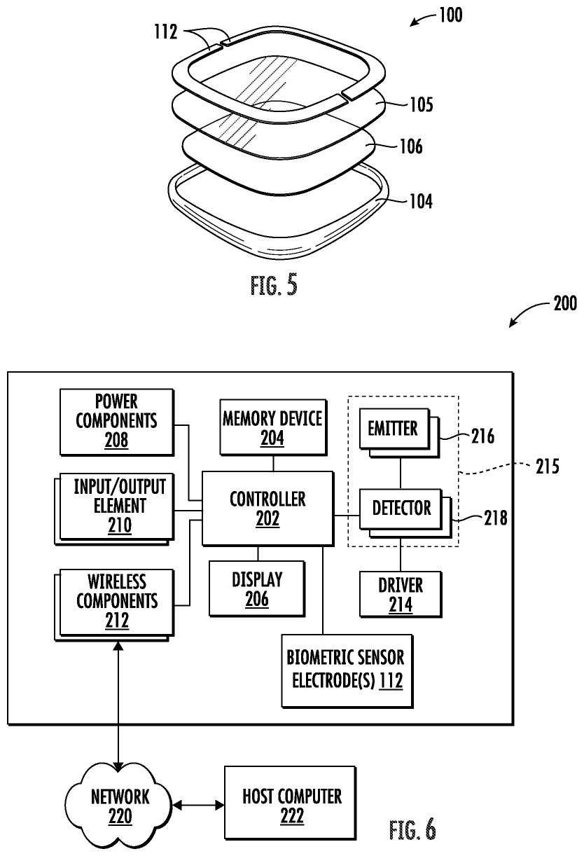

Referring now to the drawings, FIGS. 2-5 illustrate perspective views of a wearable computing device 100 according to the present disclosure. In particular, as shown in FIG. 2, the wearable computing device 100 may be worn on a user's forearm 102 like a wristwatch. Thus, as shown, the wearable computing device 100 may include a wristband 103 for securing the wearable computing device 100 to the user's forearm 102. In addition, as shown in FIGS. 2, 3, and 5, the wearable computing device 100 has an outer covering 105 and a housing 104 that contains the electronics associated with the wearable computing device 100. For example, in an embodiment, the outer covering 105 may be constructed of glass, polycarbonate, acrylic, or similar. Further, as shown in FIGS. 2, 3, and 5, the wearable computing device 100 includes an electronic display 106 arranged within the housing 104 and viewable through the outer covering 105. Moreover, as shown, the wearable computing device 100 may also include one or more buttons 108 that may be implemented to provide a mechanism to activate various sensors of the wearing computing device 100 to collect certain health data of the user. Moreover, in an embodiment, the electronic display 106 may cover an electronics package (not shown), which may also be housed within the housing 104.

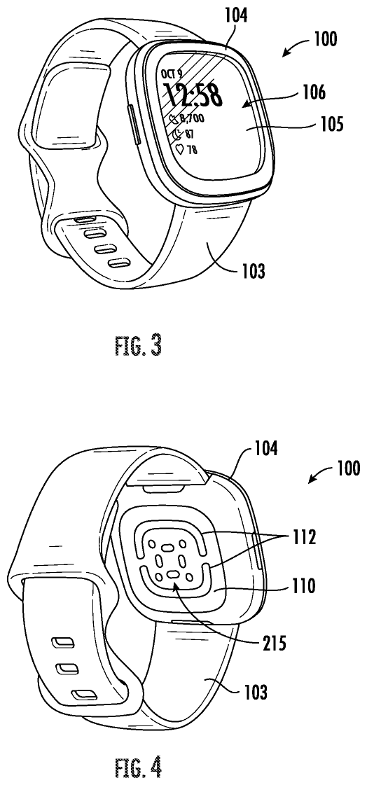

Referring particularly to FIG. 4, the housing 104 of the wearable computing device 100 further includes a dorsal wrist-side face 110 configured to sit against a dorsal wrist of a user when being worn by the user and a plurality of biometric sensor electrodes 112 positioned on the dorsal wrist-side face 110 of the housing 104 so as to maintain skin contact with the user when being worn on the wrist by the user. Thus, in such embodiments, each of the biometric sensor electrodes 112 continuously measure, at least, electrical impedance of the user at a location of the skin contact on the dorsal wrist. Accordingly, in one or more embodiments, one or more (or all) of the plurality of biometric sensor electrodes 112 may be cEDA sensor electrodes. In some embodiments, the wearable computing device 100 may also include at least one additional biometric sensor electrode in addition to the cEDA sensor electrodes. In such embodiments, the additional biometric sensor electrode may include one or more temperature sensors (such as an ambient temperature sensor or a skin temperature sensor), a humidity sensor, a light sensor, a pressure sensor, a microphone, or a PPG sensor.

Further, the biometric sensor electrodes 112 described herein may be constructed of any suitable material. For example, in an embodiment, the biometric sensor electrodes 112 described herein may be constructed of stainless steel or any other material having a suitable conductivity and/or corrosion resistance and may have an optional PVD coating, that may be 1-micrometer thick titanium nitride. In such embodiments, the PVD coating may provide a desired color to the sensor electrodes 112, thereby preventing oxidation beyond what the stainless steel already provides, and also increases durability.

In additional embodiments. PVD and surface finish can be used to increase/decrease moisture retention, which affects the cEDA signal and user comfort. In particular embodiments, the biometric sensor electrodes 112 may be formed of an alloy of tin and nickel (TiN) with a shiny or mirror surface finish. Moreover, in an embodiment, the biometric sensor electrodes 112 may be constructed of a hydrophobic material or a transparent material.

Referring now to FIG. 6, components of an example system 200 of the wearable computing device 100 that can be utilized in accordance with various embodiments are illustrated. In particular, as shown, the system 200 may also include at least one controller 202 communicatively coupled to the plurality of biometric sensor electrodes 112 for determining a skin conductance level, SCL, and/or skin conductance responses, SCRs, of the user over a certain time period using the electrical impedance of the user. Thus, in such embodiments, the biometric sensor electrodes 112 can measure voltage, current, impedance, and/or any other suitable parameters that can be used by the controller 202 for EDA application.

Moreover, in an embodiment, the controller(s) 202 may be a central processing unit (CPU) or graphics processing unit (GPU) for executing instructions that can be stored in a memory device 204, such as flash memory or DRAM, among other such options. For example, in an embodiment, the memory device 204 may include RAM, ROM, FLASH memory, or other non-transitory digital data storage, and may include a control program comprising sequences of instructions which, when loaded from the memory device 204 and executed using the controller(s) 202, cause the controller(s) 202 to perform the functions that are described herein. As would be apparent to one of ordinary skill in the art, the system 200 can include many types of memory, data storage, or computer-readable media, such as data storage for program instructions for execution by the controller or any suitable processor. The same or separate storage can be used for images or data, a removable memory can be available for sharing information with other devices, and any number of communication approaches can be available for sharing with other devices. In addition, as shown, the system 200 includes any suitable display 206, such as a touch screen, organic light emitting diode (OLED), or liquid crystal display (LCD), although devices might convey information via other means, such as through audio speakers, projectors, or casting the display or streaming data to another device, such as a mobile phone, wherein an application on the mobile phone displays the data.

The system 200 may also include one or more wireless components 212 operable to communicate with one or more electronic devices within a communication range of the particular wireless channel. The wireless channel can be any appropriate channel used to enable devices to communicate wirelessly, such as Bluetooth, cellular. NFC, Ultra-Wideband (UWB), or Wi-Fi channels. It should be understood that the system 200 can have one or more conventional wired communications connections as known in the art.

The system 200 also includes one or more power components 208, such as may include a battery operable to be recharged through conventional plug-in approaches, or through other approaches such as capacitive charging through proximity with a power mat or other such device, in further embodiments, the system 200 can also include at least one additional I/O device 210 able to receive conventional input from a user. This conventional input can include, for example, a push button, touch pad, touch screen, wheel, joystick, keyboard, mouse, keypad, or any other such device or element whereby a user can input a command to the system 200. In another embodiment, the I/O device(s) 210 may be connected by a wireless infrared or Bluetooth or other link as well in some embodiments. In some embodiments, the system 200 may also include a microphone or other audio capture element that accepts voice or other audio commands. For example, in particular embodiments, the system 200 may not include any buttons at all, but might be controlled only through a combination of visual and audio commands, such that a user can control the wearable computing device 100 without having to be in contact therewith. In certain embodiments, the I/O elements 210 may also include one or more of the biometric sensor electrodes 112 described herein, optical sensors, barometric sensors (e.g., altimeter, etc.), and the like.

Still referring to FIG. 6, the system 200 may also include a driver 214 and at least some combination of one or more emitters 216 and one or more detectors 218 (referred to herein as an optics package 215) for measuring data for one or more metrics of a human body, such as for a person wearing the wearable computing device 100. In such embodiments, as shown in FIG. 4, for example, the optics package 215 may be arranged within the housing 104 and at least partially exposed through the dorsal wrist-side face 110 of the housing 104. Thus, as shown and further explained herein, the biometric sensor electrodes 112 may be positioned around the optics package 215 on the wrist-side face 110 of the housing 104. In alternative embodiments, the various components of the optics package 215 may be positioned around the biometric sensor electrodes 112 and/or in another other suitable configuration such as adjacent to, interspersed with, surrounded by, or on top of the optics package 215. In certain embodiments, for example, wherein the biometric sensor electrodes 112 are transparent, the biometric sensor electrodes 112 may be arranged atop the optics package 215.

Referring back to FIG. 6, in some embodiments, this may involve at least one imaging element, such as one or more cameras that are able to capture images of the surrounding environment and that are able to image a user, people, or objects in the vicinity of the device. The image capture element can include any appropriate technology, such as a CCD image capture element having a sufficient resolution, focal range, and viewable area to capture an image of the user when the user is operating the device. Further image capture elements may also include depth sensors. Methods for capturing images using a camera element with a computing device are well known in the art and will not be discussed herein in detail. It should be understood that image capture can be performed using a single image, multiple images, periodic imaging, continuous image capturing, image streaming, etc. Further, the system 200 can include the ability to start and/or stop image capture, such as when receiving a command from a user, application, or other device.

The emitters 216 and detectors 218 of FIG. 6 may also be capable of being used, in one example, for obtaining optical photoplethysmogram (PPG) measurements. Some PPG technologies rely on detecting light at a single spatial location, or adding signals taken from two or more spatial locations. Both of these approaches result in a single spatial measurement from which the heart rate (HR) estimate (or other physiological metrics) can be determined. In some embodiments, a PPG device employs a single light source coupled to a single detector (i.e., a single light path). Alternatively, a PPG device may employ multiple light sources coupled to a single detector or multiple detectors (i.e., two or more light paths). In other embodiments, a PPG device employs multiple detectors coupled to a single light source or multiple light sources (i.e., two or more light paths). In some cases, the light source(s) may be configured to emit one or more of green, red, infrared (IR) light, as well as any other suitable wavelengths in the spectrum (such as long IR for metabolic monitoring). For example, a PPG device may employ a single light source and two or more light detectors each configured to detect a specific wavelength or wavelength range. In some cases, each detector is configured to detect a different wavelength or wavelength range from one another. In other cases, two or more detectors are configured to detect the same wavelength or wavelength range. In yet another case, one or more detectors configured to detect a specific wavelength or wavelength range different from one or more other detectors). In embodiments employing multiple light paths, the PPG device may determine an average of the signals resulting from the multiple light paths before determining an HR estimate or other physiological metrics.

Moreover, in an embodiment, the emitters 216 and detectors 218 may be coupled to the controller 202 directly or indirectly using driver circuitry by which the controller 202 may drive the emitters 216 and obtain signals from the detectors 218. The host computer 222 can communicate with the wireless networking components 212 via the one or more networks 220, which may include one or more local area networks, wide area networks, and/or internetworks using any of terrestrial or satellite links. In some embodiments, the host computer 222 executes control programs and/or application programs that are configured to perform some of the functions described herein.

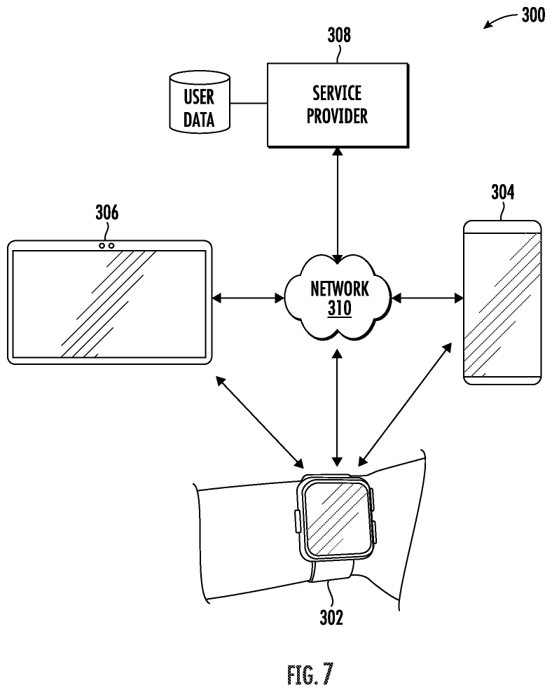

Referring now to FIG. 7, a schematic diagram of an environment 300 in which aspects of various embodiments can be implemented is illustrated. In particular, as shown, a user might have a number of different devices that are able to communicate using at least one wireless communication protocol. For example, as shown, the user might have a smartwatch 302 or fitness tracker (such as wearable computing device 100), which the user would like to be able to communicate with a smartphone 304 and a tablet computer 306. The ability to communicate with multiple devices can enable a user to obtain information from the smartwatch 302, e.g., data captured using a sensor on the smartwatch 302, using an application installed on either the smartphone 304 or the tablet computer 306. The user may also want the smartwatch 302 to be able to communicate with a service provider 308, or other such entity, that is able to obtain and process data from the smartwatch and provide functionality that may not otherwise be available on the smartwatch or the applications installed on the individual devices. In addition, as shown, the smartwatch 302 may be able to communicate with the service provider 308 through at least one network 210, such as the Internet or a cellular network, or may communicate over a wireless connection such as Bluetooth® to one of the individual devices, which can then communicate over the at least one network. There may be a number of other types of, or reasons for, communications in various embodiments.

In addition to being able to communicate, a user may also want the devices to be able to communicate in a number of ways or with certain aspects. For example, the user may want communications between the devices to be secure, particularly where the data may include personal health data or other such communications. The device or application providers may also be required to secure this information in at least some situations. The user may want the devices to be able to communicate with each other concurrently, rather than sequentially. This may be particularly true where pairing may be required, as the user may prefer that each device be paired at most once, such that no manual pairing is required. The user may also desire the communications to be as standards-based as possible, not only so that little manual intervention is required on the part of the user but also so that the devices can communicate with as many other types of devices as possible, which is often not the case for various proprietary formats. A user may thus desire to be able to walk in a room with one device and have such device automatically communicate with another target device with little to no effort on the part of the user. In various conventional approaches, a device will utilize a communication technology such as Wi-Fi to communicate with other devices using wireless local area networking (WLAN). Smaller or lower capacity devices, such as many Internet of Things (IoT) devices, instead utilize a communication technology such as Bluetooth®, and in particular Bluetooth Low Energy (BLE) which has very low power consumption.

In further embodiments, the environment 300 illustrated in FIG. 7 enables data to be captured, processed, and displayed in a number of different ways. For example, data may be captured using sensors on the smartwatch 302, but due to limited resources on the smartwatch 302, the data may be transferred to the smartphone 304 or the service provider 308 (or a cloud resource) for processing, and results of that processing may then be presented back to that user on the smartwatch 302, smartphone 304, and/or another such device associated with that user, such as the tablet computer 306. In at least some embodiments, a user may also be able to provide input such as health data using an interface on any of these devices, which can then be considered when making that determination.

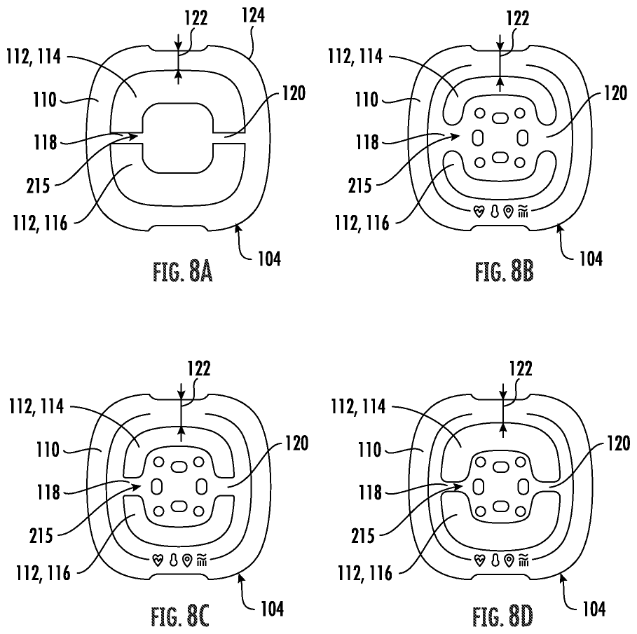

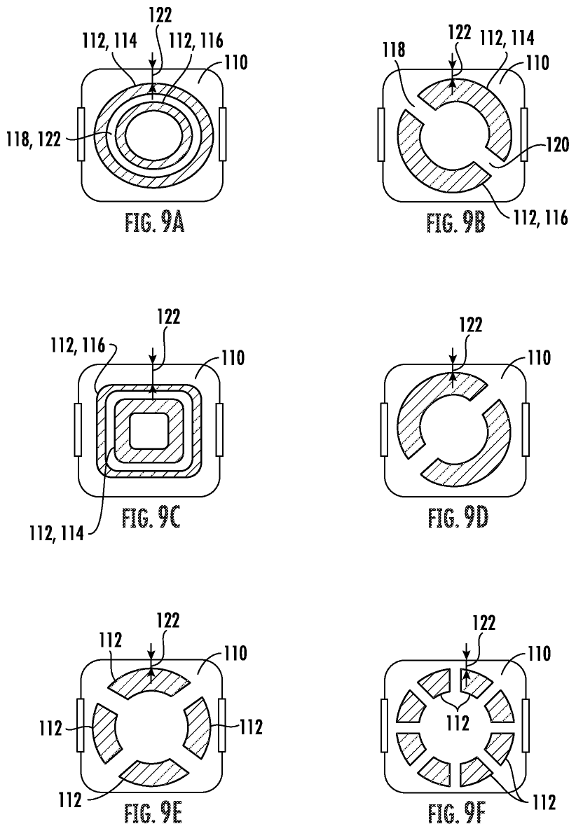

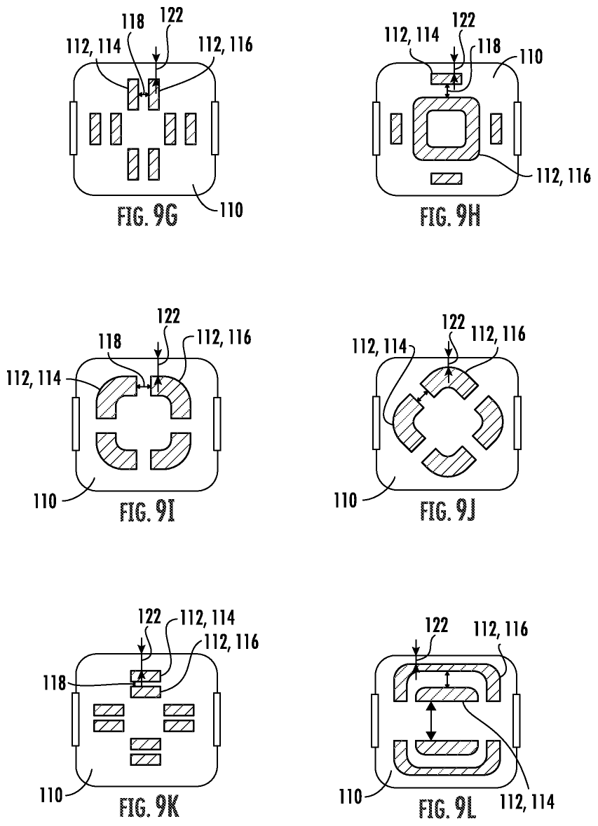

Referring now to FIGS. 8A-8D and 9A-9L, various views of multiple embodiments of the wrist-side face 110 of the housing 104 of the wearable computing device 100 according to the present disclosure are illustrated, particularly illustrating different arrangements of the biometric sensor electrodes 112 on the wrist-side face 110. It should be understood that each embodiment of the arrangement of the plurality of biometric sensor electrodes 112 described herein is not meant to be limiting and is provided herein as an example arrangement. In particular, as shown throughout FIGS. 8A-8D and 9A-9L, the plurality of biometric sensor electrodes 112 are spaced apart from an edge 124 of the wrist-side face 110 of the housing 104 by an edge gap 122 to maintain the plurality of biometric sensor electrodes 112 towards a center of the wrist-side face 110 of the housing 104, i.e., to assist in maintaining the skin contact with the user. Such arrangements provide multiple benefits, including maximizing electrode surface area, while also being as close as possible to the center of the wrist-side face 110 of the wearable computing device 100 such that skin contact is maintained with the user throughout use. Still further arrangements can also be utilized that provide the same benefits of the arrangements described herein.

In addition, as shown, two or more of the plurality of biometric sensor electrodes 112 may have different sizes and/or shapes. In particular embodiments, for example, the narrowest dimension of each of the biometric sensor electrodes 112 may range from about 2 millimeters to about 10 mm, such as about 5 mm, or about 4.5 mm. Thus, an overall area of all of the biometric sensor electrodes 112 on the wrist-side face 110 of the housing 104 may range from about 100 square millimeters (mm2) to about 150 mm2, such as about 130 mm2, In further embodiments, the plurality of biometric sensor electrodes 112 described herein may have generally curved edges rather than sharp edges, and may also be flush with the housing 104. In still further embodiments, the plurality of biometric sensor electrodes 112, which are desired to be as large as possible to maximize skin contact, provide additional benefits of larger electrode lengths and/or bonding boxes.

In further embodiments, each of FIGS. 8A and 813 illustrate a first biometric sensor electrode 114 and a second biometric sensor electrode 116. Further, as shown, the first and second biometric sensor electrodes 114, 116 are spaced apart 1w at least one gap 118, 120. In particular, as shown, the first and second biometric sensor electrodes 114, 116 are spaced apart by a first gap 118 and a second gap 120 arranged on opposing sides of the optics package 215. It should be understood that the gaps described herein may be any suitable size. For example, in particular embodiments, the gap(s) described herein may range from about 1 mm to about 10 mm, such as about 5 mm, or more preferably about 2 mm.

Referring now to FIGS. 9A-9L, various views of multiple embodiments of the wrist-side face 110 of the housing 104 of the wearable computing device 100 according to the present disclosure are illustrated, particularly illustrating different arrangements of the biometric sensor electrodes 112 on the wrist-side face 110. In particular, as shown in FIG. 9A, the first and second biometric sensor electrodes 114, 116 may be arranged in a concentric configuration. Thus, as shown, in such embodiments, the gap 118 between the first and second biometric sensor electrodes 114, 116 is an annular gap 122.

In another embodiment, as shown in FIG. 9B, the first and second biometric sensor electrodes 114, 116 may be arranged in an annular configuration around the optics package 215 (not shown). In contrast, as shown in FIGS. 9C, 9I, and 9J, each of the first and second biometric sensor electrodes 114, 116 may have a quadrilateral configuration (e.g., square, rectangular, etc.). Further, in particular embodiments, as shown in FIG. 9C, the biometric sensor electrodes 112 may be arranged such that one of the biometric sensor electrodes 114 is nested within the other (i.e., one of the biometric sensor electrodes 116 is larger than the other such that the smaller biometric sensor electrode 114 it fits within the other).

As shown in FIGS. 9E-9L, the plurality of biometric sensor electrodes 112 may include more than two biometric sensor electrodes arranged around the optics package 215. For example, as shown in FIGS. 9E and 9F, the wearable computing device 100 may have more than two biometric sensor electrodes 114, 116 arranged in an annular configuration around the optics package 215 (not shown) More specifically, as shown in FIG. 9E, four biometric sensor electrodes 112 are arranged on the wrist-side face 110 in an annular configuration. In another embodiment, as shown in FIG. 9F, eight biometric sensor electrodes 112 are arranged on the wrist-side face 110 in an annular configuration. One having ordinary skill in the art would appreciate that any number of biometric sensor electrodes may be arranged on the wrist-side face 110 of the wearable computing device 100 so as to increase surface area of the sensor electrodes, thereby improving sensor signal quality.

Referring now to FIGS. 9G, 9H, and 9K, at least two of the plurality of biometric sensor electrodes 112 may be arranged in pairs 114, 116. For example, the controller(s) 202 is configured to select one of the pairs 114, 116 of the plurality of biometric sensor electrodes 112 for determining the SCL of the user over the certain time period based upon data collected therefrom. In certain embodiments, for power savings and device compactness, the biometric sensor electrodes 112 may be duty cycled (i.e. turned off intermittently). Even with only one pair of electrodes active at a time, the wearable computing device 100 can still achieve continuous measurement across every possible pair of electrodes by rapidly switching the active pair. This reduces the power budget, avoids contamination of the signal between different electrode pairs, and allows the controller to select the optimal electrode pair to output data from. Alternatively, multiple pairs of biometric sensor electrodes can be enabled with, e.g., non-overlapping square wave excitation, meaning that there is nearly constant excitation current, but at any given moment it is only coining from a single electrode pair.

Further, in such embodiments, as shown, at least a portion of each of the pairs 114, 116 of biometric sensor electrodes 112 may be parallel to each other and spaced apart by a gap 118 of a certain distance. Thus, such an arrangement can be beneficial for receiving sample data from multiple, but similarly located sensor electrodes such that the best or most accurate data collected between the two biometric sensor electrodes 114, 116 can be used for further processing. In particular, as shown in FIGS. 9G and 9K, the pairs 114, 116 of biometric sensor electrodes 112 may have substantially the same size and/or dimension and may be arranged in a vertical direction with respect to the wrist-side face 110 (FIG. 9G), in a horizontal direction with respect to the wrist-side face 110 (FIG. 9k), and/or combinations thereof. In alternative embodiments, as shown in 9H, the biometric sensor electrodes 112 may include a larger, central biometric sensor electrode 116 with a plurality of smaller, biometric sensor electrodes 114 surrounding the larger, central biometric sensor electrode 116.

Referring to FIG. 9L, in yet another embodiment, the plurality of biometric sensor electrodes 112 may be arranged similar to FIG. 9C, but with multiple gaps 118, 120 therebetween. For example, as shown, the plurality of biometric sensor electrodes 112 may include two sets of a first biometric sensor electrode 114 and a second biometric sensor electrode 116. Each set is arranged on opposing sides of the optics package 215 (not shown) and is separated by gap 120. Further, each of the first and second biometric sensor electrodes 114, 116 are also separated by gap 118.

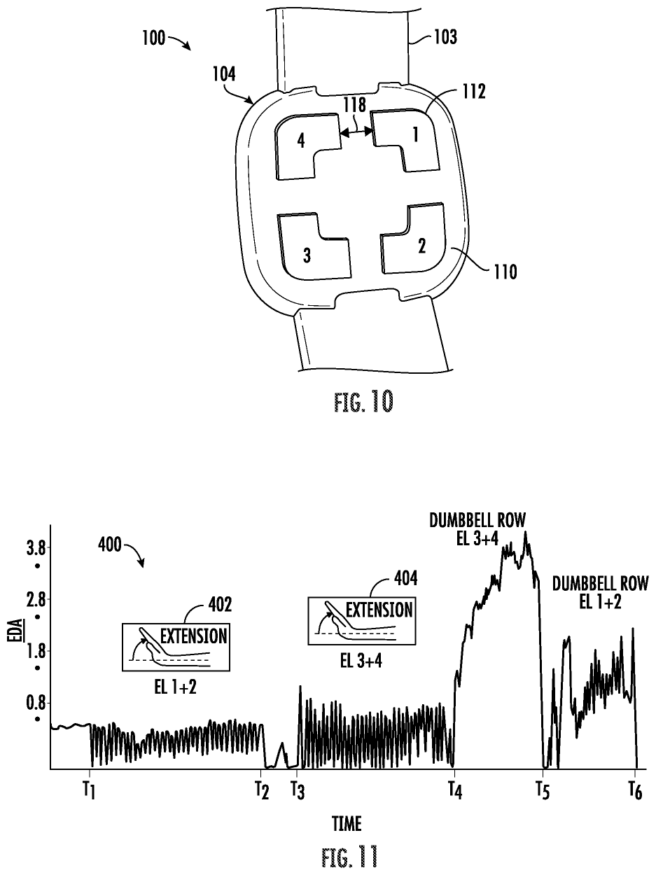

Referring now to FIG. 10, in still another embodiment, one or more of the plurality of biometric sensor electrodes 112 may be elevated with respect to an area adjacent to the plurality of biometric sensor electrodes 112 on a surface of the wrist-side face 110 of the housing 104. In such embodiments, as shown, the biometric sensor electrodes 112 may be raised with respect to the wrist-side face 110 of the housing 104. In further embodiments, the biometric sensor electrodes 112 may sit flush with the wrist-side face 110 of the housing 104, but may include a channel or recess adjacent thereto. Such arrangements are generally efficient for allowing evaporation directly dissipating into the surrounding air. In such embodiments, the channel(s) may be configured to connect to the outside air.

Referring now to FIGS. 10 and 11 one embodiment of an arrangement of the biometric sensor electrodes 112 (formed of stainless steel 316 L electrodes) with respect to a user's elbow and hand is provided along with a graph 400 of cEDA measurements of the biometric sensor electrodes 112 with respect to time is illustrated. In particular, as shown between T1 and T2, cEDA measurements from biometric sensor electrodes 1 and 2 (FIG. 10) are provided when a user rotates his or her hand wrist as shown in the onset 402. Further, as shown between T3 and T4, cEDA measurements from biometric sensor electrodes 3 and 4 (FIG. 10) are provided when a user rotates his or her hand wrist as shown in the onset 404, Moreover, as shown between T4 and T5, cEDA measurements from biometric sensor electrodes 3 and 4 (FIG. 10) are provided when a user completes a dumbbell row. In addition, as shown between T5 and T6, cEDA measurements from biometric sensor electrodes 1 and 2 (FIG. 10) are provided when a user completes a dumbbell row. Thus, FIGS. 10 and 11 provide a real-life example of how cEDA measurements using multiple single-path electrodes present opportunities for evaluating electrode positioning and gesturing/exercise (e.g., weights), Moreover, in particular embodiments, certain electrode combinations may have optimized cEDA signal quality (i.e., with respect to subject/activity variances).

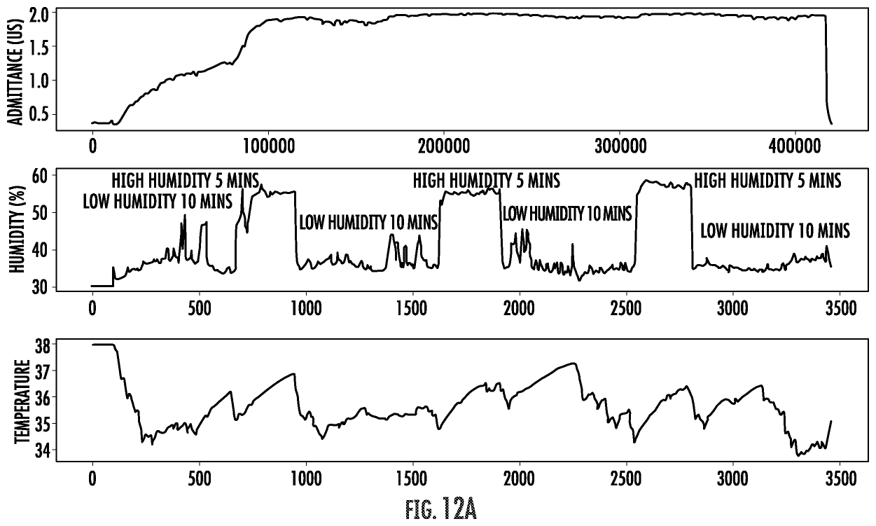

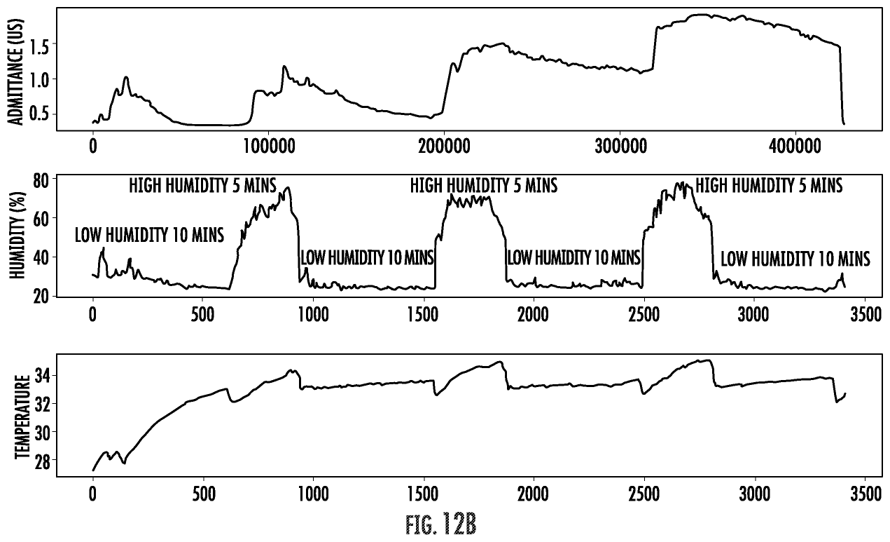

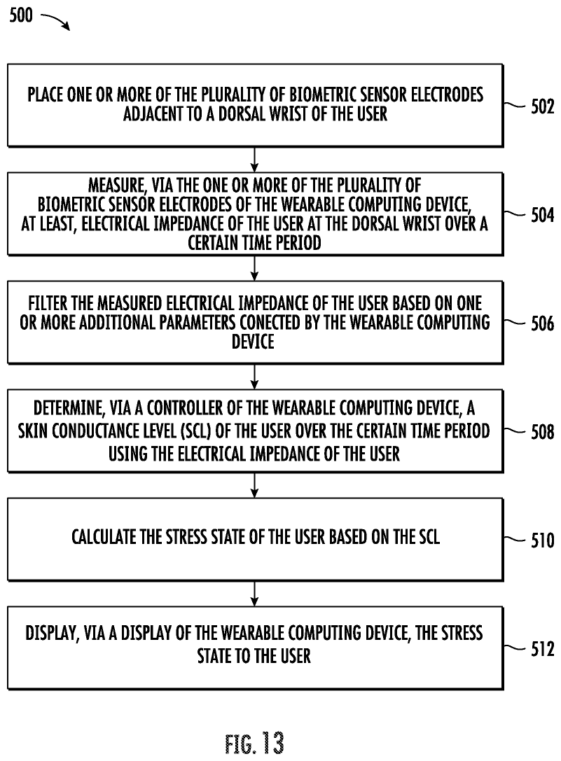

Referring now to FIGS. 12A and 12B, graphs of temperature (e.g. such as skin and/or ambient temperature), humidity, and conductivity of a user wearing the wearable computing device 100 described herein are provided according to the present disclosure. In particular, as shown in FIG. 12A, the user moves between two rooms, one with a humidifier and the other without. Further, as shown in FIG. 12B, the user moves between two rooms, one using a hot shower to increase humidity and the other without. Thus, as generally shown in the graphs of FIGS. 12A and 12B, the presence of nuisance variables (e.g., ambient and subject conditions), render data insufficient for drawing clear relationships between cEDA, temperature, and humidity. Thus, the additional temperature and/or humidity sensors described herein can be beneficial in removing such nuisance data to further improve cEDA measurements of the user.

Referring now to FIG. 13, a flow diagram of one embodiment of a method 500 of monitoring a stress state of a user using a wearable computing device is provided. In an embodiment, for example, the wearable computing device may be any suitable wearable computing device such as the wearable computing device 100 described herein with reference to FIGS. 1-10. In general, the method 500 is described herein with reference to the wearable computing device 100 of FIGS. 1-10. However, it should be appreciated that the disclosed method 500 may be implemented with any other suitable wearable computing device having any other suitable configurations. In addition, although FIG. 13 depicts steps performed in a particular order for purposes of illustration and discussion, the methods discussed herein are not limited to any particular order or arrangement. One skilled in the art, using the disclosures provided herein, will appreciate that various steps of the methods disclosed herein can be omitted, rearranged, combined, and/or adapted in various ways without deviating from the scope of the present disclosure.

As mentioned and described herein, the wearable computing device includes a plurality of biometric sensor electrodes on a dorsal wrist-side face of a housing of the wearable computing device. Thus, as shown at (502), the method 500 includes placing one or more of the plurality of biometric sensor electrodes adjacent to a dorsal wrist of the user. For example, in an embodiment, the method 500 may include arranging the plurality of biometric sensor electrodes around, adjacent to, interspersed with, surrounded by, or on top of an optics package on the dorsal wrist-side face of the housing. As shown at (504), the method 500 includes continuously measuring, via the one or more of the plurality of biometric sensor electrodes of the wearable computing device, at least, one or more parameters indicative of electrical impedance of the user at the wrist over a certain time period. As shown at (506) the method 500 includes filtering the measured electrical impedance of the user based on one or more additional parameters collected by the wearable computing device. For example, as explained with respect to FIGS. 12A and 12B, certain parameters or events (such as humidity, temperature, conductivity, noise, pressure, light, etc.) can be considered and removed from the collected data.

Referring back to FIG. 13, as shown at (508), the method 500 includes determining, via at least one controller of the wearable computing device, a skin conductance level, SCL, of the user over the certain time period based on the electrical impedance of the user. As shown at (510), the method 500 includes calculating, via the controller(s), the stress state of the user based on the SCL or a combination of SCL with other device-collected metrics, such as heart rate. As shown at (512), the method 500 includes displaying, via a display of the wearable computing device, the stress state to the user. In still another embodiment, the method 500 may include selecting an optimal pair of the plurality of biometric sensor electrodes 112 for measurement, e.g., to maximize measurement value with respect to power use. As more electrodes consume more power, both for the stimulation current and signal processing, it can be valuable to have measurements from multiple paths simultaneously.

### Additional Disclosure

The technology discussed herein makes reference to servers, databases, software applications, and other computer-based systems, as well as actions taken and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure cover such alterations, variations, and equivalents.

What is claimed is:

1. A wearable computing device, comprising:
a housing comprising a dorsal wrist-side face configured to sit against a dorsal wrist of a user of the wearable computing device when being worn by the user;
an electronic display arranged within the housing;
a plurality of biometric sensor electrodes positioned on the dorsal wrist-side face of the housing so as to maintain skin contact with the user when being worn on the dorsal wrist by the user, the plurality of biometric sensor electrodes measuring, at least, one or more parameters indicative of electrical impedance of the user at a location of the skin contact;
at least one driver communicatively coupled to the plurality of biometric sensor electrodes; and
at least one controller communicatively coupled to the at least one driver, the at least one controller configured to determine a skin conductance level, SCL, of the user over a certain time period based on the electrical impedance of the user and calculate a stress state of the user based, at least in part, on the SCL.

2. The wearable computing device of claim 1, wherein each of the plurality of biometric sensor electrodes comprises continuous electrodermal activity, cEDA, sensor electrodes, the cEDA sensor electrodes configured to measure the SCL and skin conductance responses, SCRs.

3. The wearable computing device of claim 1, further comprising an optics package arranged within the housing and at least partially exposed through the dorsal wrist-side face of the housing, the plurality of biometric sensor electrodes being positioned around, adjacent to, interspersed with, surrounded by, or on top of the optics package on the dorsal wrist-side face of the housing.

4. The wearable computing device of claim 3, wherein the plurality of biometric sensor electrodes comprises, at least, a first biometric sensor electrode and a second biometric sensor electrode, the first and second biometric sensor electrodes being spaced apart by at least one gap.

5. The wearable computing device of claim 4, wherein the at least one gap comprises a first gap and a second gap arranged on opposing sides of the optics package.

6. The wearable computing device of claim 4, wherein the first and second biometric sensor electrodes are arranged in a concentric configuration, wherein the at least one gap is an annular gap.

7. The wearable computing device of claim 3, wherein the plurality of biometric sensor electrodes comprises more than two biometric sensor electrodes arranged around, adjacent to, interspersed with, surrounded by, or on top of the optics package.

8. The wearable computing device of claim 7, wherein the more than two biometric sensor electrodes are arranged in an annular configuration around the optics package.

9. The wearable computing device of claim 7, wherein the more than two biometric sensor electrodes are arranged in a quadrilateral configuration around, adjacent to, interspersed with, surrounded by, or on top of the optics package.

10. The wearable computing device of claim 1, wherein at least two of the plurality of biometric sensor electrodes are arranged in pairs, each of the pairs being parallel to each other and spaced apart by a gap.

11. The wearable computing device of claim 10, wherein the at least one controller is configured to select one of the pairs of the plurality of biometric sensor electrodes for determining the SCL of the user over the certain time period based upon data collected from the pairs of the plurality of biometric sensor electrodes.

12. The wearable computing device of claim 1, wherein two or more of the plurality of biometric sensor electrodes have different shapes.

13. The wearable computing device of claim 1, wherein the plurality of biometric sensor electrodes are spaced apart from an edge of the dorsal wrist-side face of the housing by a gap.

14. The wearable computing device of claim 1, wherein one or more of the plurality of biometric sensor electrodes is elevated with respect to an area adjacent to the plurality of biometric sensor electrodes on a surface of the dorsal wrist-side face of the housing.

15. The wearable computing device of claim 1, further comprising at least one additional biometric sensor electrode, the at least one additional biometric sensor electrode comprising at least one of one or more temperature sensors, a humidity sensor, a light sensor, a pressure sensor, a microphone, or a photoplethysmogram (PPG) sensor.

16. The wearable computing device of claim 1, wherein one or more of the plurality of biometric sensor electrodes comprises at least one of the following characteristics: transparency, flushness with the dorsal wrist-side face, a surface finish, or curved edges.

17. A computer-implemented method of monitoring a stress state of a user using a wearable computing device, the wearable computing device having a plurality of biometric sensor electrodes on a dorsal wrist-side face of a housing of the wearable computing device, the computer-implemented method comprising:
placing one or more of the plurality of biometric sensor electrodes adjacent to a dorsal wrist of the user;
continuously measuring, via the one or more of the plurality of biometric sensor electrodes of the wearable computing device, at least, one or more parameters indicative of electrical impedance of the user at the wrist over a certain time period;
determining, via a controller of the wearable computing device, a skin conductance level, SCL, of the user over the certain time period based on the electrical impedance of the user;
calculating, via the controller, the stress state of the user based, at least in part, on the SCL; and
displaying, via a display of the wearable computing device, the stress state to the user.

18. The computer-implemented method of claim 17, further comprising selecting an optimal pair of electrodes for measurement.

19. The computer-implemented method of claim 17, further comprising filtering the electrical impedance of the user based on one or more additional parameters collected by the wearable computing device.

20. The computer-implemented method of claim 17, wherein the plurality of biometric sensor electrodes are arranged around, adjacent to, interspersed with, surrounded by, or on top of an optics package on the dorsal wrist-side face of the housing.
